(19) United States
(12) Patent Application Publication
(10) Pub. No.: US 2022/0015394 A1
(43) Pub. Date: Jan. 20, 2022

(54) Use of An Enzyme Granule

(71) Applicant: Novozymes A/S, Bagsvaerd (DK)

(72) Inventors: Albert E. Cerrera-Padrell, Frederiksberg (DK); Niels-Viktor Nielsen, Saaby (DK)

(73) Assignee: Novozymes A/S, Bagsvaerd (DK)

(21) Appl. No.: 17/296,062

(22) PCT Filed: Dec. 5, 2019
(86) PCT No.: PCT/EP2019/083765

§ 371 Date: May 21, 2021

(30) Foreign Application Priority Data
Dec. 5, 2018 (EP) 18210418.2

(51) Int. Cl. A23K 20/189; A23K 20/111; A23K 20/158; A23K 20/163; A23K 40/30; C12N 9/98; C12N 9/64; A23K 40/10

(52) U.S. Cl. CPC A23K 20/189; A23K 20/111; A23K 20/158; A23K 40/10; A23K 40/30; C12N 9/98; C12N 9/6408; A23K 20/163

(57) ABSTRACT

The present invention relates to the use of low dust enzyme granules for post pelleting liquid application (PPLA) or liquid application on other types of non-pelleted feed, such as mash feed. The invention further relates to a process for producing the low dust enzyme granules for liquid application.

### FIELD OF THE INVENTION

The present invention relates to the use of low dust enzyme granules for post pelleting liquid application (PPLA) or liquid application on other types of non-pelleted feed, such as mash feed. The invention further relates to a process for producing the low dust enzyme granules for liquid application.

### BACKGROUND OF THE INVENTION

Animal feed containing ingredients such as vitamins, amino acids, minerals and enzymes is typically provided as feed pellets. The pellets are prepared at feed pelleting mills operating at temperatures above 70° C.-80° C. to avoid growth of bacteria and improve pellet quality and digestibility. Ingredients, such as enzymes are typically added to the feed mill as solid ingredients. However, enzymes may be heat sensitive and may not survive the heat treatment. Also, a problem with many solid ingredients, in particular enzymes, is that they tend to form dust during physical handling, e.g., during processing in mixing and packaging machines, or even after crushing of spilled particles by equipment, shoes or wheels. This not only creates waste product, but the dust can also cause serious hygiene and health problems.

To overcome this, enzymes may be added to feed pellets through post pelleting liquid application (PPLA). The enzymes are typically applied onto the heat-treated pellets as a liquid composition by spraying at the die, spraying into a screw conveyor, spraying into a plenum or weir or spraying using spinning disks to atomize the liquid. Enzymes can also be added as a liquid composition to other types of feed, such as non-pelletized mash feed. Traditionally, such direct application of enzymes onto mash feed or feed pellets has been done from enzyme liquid compositions. While liquid compositions have the inherent advantage of suppressing enzyme dust formation, they have several disadvantages compared to solid formulations, such as poorer stability. WO 09/102770 describes enzyme-containing granules with a diameter of about 150 to about 355 microns comprising a single core and an enzyme-containing layer coated over the core, where the core consists of one or more inorganic salts. WO 06/034710 describes steam treated pelletized feed composition comprising a granule comprising a core and a coating wherein the core comprises an active compound and the coating comprises a salt. WO 07/044968 discloses granules for feed compositions comprising: a core, an active agent, and at least one coating, where the granules are particularly suitable for inclusion in steam treatment processes, including pelleting and tableting processes and steam processing of feed, without appreciable loss of active agent activity. WO9739116 relates to an enzyme-containing granule comprising an enzyme and a core capable of absorbing at least 5% water. WO 17/162610 relates to enzyme compositions in a dry form which comprise one or more water-soluble feed enzymes, a salt of benzoic acid and a weak acid and to the use of these enzyme compositions to prepare the enzyme compositions in liquid form.

WO 05/074707, WO 18/007154, WO 2009/152176 disclose different liquid enzyme formulations.

Enzymes stored as liquid compositions require large storing facilities and are generally less stable than enzymes in dry form. Enzymes in dry form such as lyophilized or spray dried enzymes often have a tendency of forming dust. Thus, there is a need for enzyme compositions for use in post pelleting liquid application (PPLA) or other liquid applications on feed.

### SUMMARY OF THE INVENTION

The invention provides for the use of low dust enzyme granules for post pelleting liquid application (PPLA) or liquid application on other types of non-pelleted feed, such as mash feed, of at least one enzyme, wherein the enzyme granule is dissolved in water before application.

The dissolved granules for use in the invention may be applied onto pellets or mash feed as a liquid composition by spray. In one aspect of the invention, the granules are dissolved and sprayed onto feed pellets in a feed mill.

The invention further provides a process for producing low dust enzyme granules for the use in liquid application, where the process comprises preparing a granule comprising a core and at least one enzyme wherein the enzyme is distributed in the core and/or layered over the core, and applying to the core or layered granule an outer layer to obtain a coated granule. In one aspect of the invention, the granules are prepared in a fluid bed apparatus.

### DETAILED DESCRIPTION OF THE INVENTION

Definitions

Animal feed: The term “animal feed” refers to any compound, preparation, or mixture suitable for, or intended for intake by an animal. Animal feed for a mono-gastric animal typically comprises concentrates as well as vitamins, minerals, enzymes, direct fed microbial, amino acids and/or other feed ingredients (such as in a premix) whereas animal feed for ruminants generally comprises forage (including roughage and silage) and may further comprise concentrates as well as vitamins, minerals, enzymes direct fed microbial, amino acid and/or other feed ingredients (such as in a premix).

Dust: The term “dust” in connection with granules or powders refers to the tendency of a granule or powder, upon handling, to liberate fine airborne particles. Granule or powder dust is routinely measured in the industry and may be measured by several different techniques. Well known methods for measuring enzyme dust e.g. include the Elutriation assay and the Heubach Type 1 assay.

Enzyme: The enzyme in the context of the present invention may be any enzyme or combination of different enzymes. Accordingly, when reference is made to “an enzyme” this will in general be understood to include one enzyme or a combination of enzymes. It is to be understood that enzyme variants (produced, for example, by recombinant techniques) are included within the meaning of the term “enzyme”. Examples of such enzyme variants are disclosed, e.g. in EP 251,446 (Genencor), WO 91/00345 (Novo Nordisk), EP 525,610 (Solvay) and WO 94/02618 (Gist-Brocades NV).

Low dust enzyme granule: “Low dust enzyme granule” is herein used for an enzyme granule which results in little or no total dust release during handling (i.e. the enzyme granule has a low tendency of forming dust from the active and the non-active granule ingredients) when measured by the Heubach Type 1 assay or the Elutriation assay as described in the Analytical Method section under “Total dust determined by Heubach Type 1”, respectively “Total dust determined by Elutriation”. In one aspect, the dust is below 1000 μg/g in Heubach Type 1 assay and/or below 1000 μg/g in Elutriation assay. In a further aspect, the dust is below 500 μg/g in Heubach Type 1 assay, below 250 μg/g in Heubach Type 1 assay, below 100 μg/g in Heubach Type 1 assay or below 50 μg/g in Heubach Type 1 assay. In a yet further aspect, the dust is below 500 μg/g in Elutriation assay, below 250 μg/g in Elutriation assay, below 100 μg/g in Elutriation assay or below 50 μg/g in Elutriation assay.

Low active dust enzyme granule: Is a Low dust enzyme granule which results in little or no active enzyme dust fraction when measured by the Heubach Type 1 assay or the Elutriation assay as described in the Analytical Method section under “Active dust fraction determined by Heubach Type 1 dust-meter”, respectively “Active dust fraction determined by Elutriation”. In one aspect, the active dust fraction is below 20 ppm in Heubach Type 1 assay and/or below 80 ppm in Elutriation assay. In a further aspect, the active dust fraction is below 10 ppm in Heubach Type 1 assay, below 6 ppm, below 2 ppm or below 0.5 ppm in Heubach Type 1 assay. In a yet further aspect, the active dust fraction is below 40 ppm in Elutriation assay, below 20 ppm, below 10 ppm, below 4 ppm, below 2 ppm or below 0.5 ppm in Elutriation assay.

Inert material: Inert material is material that is not chemically reactive. Examples of inert material is e.g. salts such as sodium sulfate, sodium chloride or carbohydrate.

Particle Size Distribution (PSD): The term “Particle Size Distribution” or “PSD” is herein used for granules of the invention and defines the relative amount, typically by volume, of particles present according to size. The PSD is described as the D-Values D10, D50 and D90, wherein D10 refers to the 10% percentile of the particle size distribution (meaning that 10% of the volume of the particles has a size equal or less than the given value), D50 describes the 50% percentile and D90 describes the 90% percentile. Particle size distribution may be measured using laser diffraction methods or optical digital imaging methods or sieve analysis. D-Values reported herein were measured by laser diffraction, where the particle size was reported as a volume equivalent sphere diameter.

Pellet: The terms “pellet” and/or “pelleting” refer to solid rounded, spherical and/or cylindrical tablets or pellets and the processes for forming such solid shapes, particularly feed pellets and solid extruded animal feed. As used herein, the terms “extrusion” or “extruding” are terms well known in the art and refer to a process of forcing a composition, as described herein, through an orifice under pressure.

Percentage (%): When used herein “%” means weight percentage, also sometimes written as w/w. For example, when written that the granule comprises at least 10% active enzyme it means that 10% of the weight of the granule is active enzyme.

Post pelleting liquid application (PPLA): Post pelleting liquid application (PPLA) is the addition of ingredients such as e.g. fat, vitamins, enzymes and/or probiotics from a liquid composition to feed pellets after the pellets have been prepared by a steam-heated pelleting process.

The Invention

With the present invention we describe the use of low dust enzyme granules for post pelleting liquid application (PPLA) to feed pellets or liquid application on other types of non-pelleted feed, such as mash feed, where the enzyme granules are dissolved in water before the application.

The enzyme granules disclosed herein are particularly suited for the use because they are low dust enzyme granules and thus safer to handle, they are easy to handle and easy to transport. The enzyme granules for use in the invention have a high density and a high content of enzyme. In one aspect of the invention, the enzyme granules have a bulk density which is at least 0.6 g/mL. In another aspect of the invention, the enzyme granules have a content of active enzyme of at least 10% w/w, preferably at least 20% w/w, and even more preferably at least 30% w/w. High bulk density and high active enzyme content are an advantage for e.g. high value compaction, lower transportation and packaging costs.

Furthermore, the enzyme granules for use in the invention have an excellent flowability, which can be measured by methods known by the person skilled in the art, e.g. by measuring angle of repose. The precision of the dosing performed by a mechanical dispenser system depends on the flowability of the product. Cohesive products will often break up in lumps whereby the weight target easily is overflown. Products that segregate from the mechanical handling e.g. vibration conveyers may lead to variations in the Particle Size Distribution (PSD) that is dosed to the individual charges produced by the dispenser. Low bulk density particles in particular in combination with a wide PSD may flow too easy at the level of mechanical impact needed for handling the smaller particles and lead to overflow.

The flowability of a powder or granule is heavily influenced by its particle size distribution. Small sized particles tend to flow poorer compared to bigger particles. Small sized particles with good flowability tend to form dust. It is possible to reduce dust by adding so-called de-dusting or agglomerating agents, however typically at a cost of affecting flowability. The granules for use according to the invention have an advantageous PSD.

A further advantage of the enzyme granules is their quick dissolution profile. In one aspect of the invention, no precipitates are seen after the granules are dissolved in water. In a further aspect of the invention, the solubility of the granules is determined by a) dissolving the granules at 1.5% concentration in water, b) sieving the solution from step a) through a 100 micrometer sieve, c) drying the sieve and d) checking the weight of insoluble matter captured by the sieve, wherein the granules are soluble in water if there is less than 0.5% residual matter. A yet further advantage is that the granules are stable. In one aspect of the invention, the enzymes of the granules are active for at least 12 hours after dissolution in water, in a further aspect, the enzymes of the granules are active for at least 16 hours after dissolution. In a preferred aspect, the enzymes of the granules are active for at least 24 hours after dissolution in water. In one aspect, the granules are physically stable. In a further aspect of the invention wherein the granules are physically stable, precipitates are not formed after 24 hours upon dissolution in water at 30° C. In one aspect, the granules are enzymatically stable. In a further aspect wherein the granules are enzymatically stable, the enzyme activity is at least 95% of the initial enzyme activity after 24 hours upon dissolution in water at 30° C. In one aspect, the granules have microbial stability. In a further aspect, the granules have microbial stability according to the requirements of the U.S. Food and Drug Administration (FDA). In one aspect of the invention, microbial stabilizers are introduced into the granule. In a further aspect, one or more microbial stabilizers are introduced into the granule wherein the enzyme content is contained compared to a granule without the microbial stabilizer(s).

The Granule

The enzyme granule for use according to the invention may have a matrix structure where the components have been mixed homogeneously. Alternatively, the enzyme granule comprises a core particle and one or more coatings, such as e.g. salt and/or wax coatings, where the core particle either comprises an enzyme, optionally as a blend of one or more enzymes with one or more salts or additives, or an inert particle with the one or more enzymes applied onto it.

Examples of wax coatings are polyethylene glycols, polypropylenes, Carnauba wax, Candelilla wax, bees wax, hydrogenated plant oil or animal tallow such as hydrogenated ox tallow, hydrogenated palm oil, hydrogenated cotton seeds and/or hydrogenated soy bean oil, fatty acid alcohols, mono-glycerides and/or di-glycerides, such as glyceryl stearate, wherein stearate is a mixture of stearic and palmitic acid, micro-crystalline wax, paraffin's, and fatty acids, such as hydrogenated linear long chained fatty acids and derivatives thereof. Other examples include polymer coatings such as e.g. described in WO 2001/00042. A preferred wax is palm oil or hydrogenated palm oil.

Examples of salt coatings are Na₂SO₄, K₂SO₄, MgSO₄, sodium citrate and mixtures of salts. Other examples are those described in e.g. WO 2008/017659, WO 2006/034710, WO 1997/05245, WO 1998/54980, WO 1998/55599, WO 2000/70034. The salt coating is typically at least 1 μm thick.

In an aspect, the core particles comprise an inert material which is selected from the group consisting of organic or inorganic salts (such as calcium acetate, calcium benzoate, calcium carbonate, calcium chloride, calcium citrate, calcium sorbate, calcium sulfate, potassium acetate, potassium benzoate, potassium carbonate, potassium chloride, potassium citrate, potassium sorbate, potassium sulfate, sodium acetate, sodium benzoate, sodium carbonate, sodium chloride, sodium citrate, sodium sulfate, zinc acetate, zinc benzoate, zinc carbonate, zinc chloride, zinc citrate, zinc sorbate, zinc sulfate), starch, sugars, carbohydrate (such as e.g. sucrose, dextrin, glucose, lactose, sorbitol), small organic molecules, starch, flour, cellulose and minerals and clay minerals (also known as hydrous aluminium phyllosilicates) and mixtures thereof. In a preferred aspect, the core comprises an inorganic salt such as sodium sulfate or sodium chloride.

In an alternative or further aspect, the core particles comprise a microbial stabilizer which is selected from the group consisting of: sorbic acid, ascorbic acid, citric acid, benzoic acid, a salt of sorbic acid, a salt of ascorbic acid, a salt of citric acid, a salt of benzoic acid, potassium sorbate, sodium citrate, sodium benzoate and combinations thereof.

In an aspect, the solid composition is in granulated form and comprises a core particle, an enzyme layer comprising one or more enzymes and a salt coating.

In a further aspect, the granule comprises a formulating agent which is selected from one or more of the following compounds: glycerol, ethylene glycol, 1, 2-propylene glycol or 1,3-propylene glycol, or other polyols, sodium chloride, sodium benzoate, potassium sorbate, sodium sulfate, potassium sulfate, magnesium sulfate, sodium thiosulfate, calcium carbonate, sodium citrate, dextrin, glucose, sucrose, sorbitol, lactose, starch or other carbohydrates, kaolin and cellulose. In a preferred aspect, the formulating agent is selected from one or more of the following compounds: 1, 2-propylene glycol, 1, 3-propylene glycol, sodium sulfate, dextrin, cellulose, sucrose, sodium thiosulfate, kaolin and calcium carbonate.

In an aspect, the granule comprises an enzyme stabilizer. In a further aspect, the granule comprises zinc or magnesium as enzyme stabilizer. In a yet further aspect, the granule comprises a magnesium salt or a zinc salt such as e.g. magnesium sulfate and zinc sulfate.

The Enzyme

It is to be understood that enzyme variants (produced, for example, by recombinant techniques) are included within the meaning of the term “enzyme”. Examples of such enzyme variants are disclosed, e.g. in EP 251,446 (Genencor), WO 91/00345 (Novo Nordisk), EP 525,610 (Solvay) and WO 94/02618 (Gist-Brocades NV).

Enzymes can be classified on the basis of the handbook Enzyme Nomenclature from NCIUBMB, 1992), see also the ENZYME site at the internet: http://www.expasy.ch/enzyme/. ENZYME is a repository of information relative to the nomenclature of enzymes. It is primarily based on the recommendations of the Nomenclature Committee of the International Union of Biochemistry and Molecular Biology (IUB-MB), Academic Press, Inc., 1992, and it describes each type of characterized enzyme for which an EC (Enzyme Commission) number has been provided (Bairoch A. The ENZYME database, 2000, Nucleic Acids Res 28:304-305). This IUBMB Enzyme nomenclature is based on their substrate specificity and occasionally on their molecular mechanism; such a classification does not reflect the structural features of these enzymes.

Another classification of certain glycoside hydrolase enzymes, such as endoglucanase, xy-lanase, galactanase, mannanase, dextranase and alpha-galactosidase, in families based on amino acid sequence similarities has been proposed a few years ago. They currently fall into 90 different families: See the CAZy(ModO) internet site (Coutinho, P.M. & Henrissat, B. (1999) Carbohydrate-Active Enzymes server at URL: http://afmb.cnrs-mrs.fr/˜cazy/CAZY/index.html (corresponding papers: Coutinho, P.M. & Henrissat, B. (1999) Carbohydrate-active enzymes: an integrated database approach. In “Recent Advances in Carbohydrate Bioengineering”, H. J. Gilbert, G. Davies, B. Henrissat and B. Svensson eds., The Royal Society of Chemistry, Cambridge, pp. 3-12; Coutinho, P.M. & Henrissat, B. (1999) The modular structure of cellulases and other carbohydrate-active enzymes: an integrated database approach. In “Genetics, Biochemistry and Ecology of Cellulose Degradation”., K. Ohmiya, K. Hayashi, K. Sakka, Y. Kobayashi, S. Karita and T. Kimura eds., Uni Publishers Co., Tokyo, pp. 15-23).

The types of enzymes which may be incorporated in granules of the invention include oxidoreductases (EC 1.-.-.-), transferases (EC 2.-.-.-), hydrolases (EC 3.-.-.-), lyases (EC 4.-.-.-), isomerases (EC 5.-.-.-) and ligases (EC 6.-.-.-).

Preferred oxidoreductases in the context of the invention are peroxidases (EC 1.11.1), laccases (EC 1.10.3.2) and glucose oxidases (EC 1.1.3.4). An Example of a commercially available oxidoreductase (EC 1.-.-.-) is GluzymeTM (enzyme available from Novozymes A/S).

Preferred hydrolases in the context of the invention are: carboxylic ester hydrolases (EC 3.1.1.-) such as lipases (EC 3.1.1.3); phytases (EC 3.1.3.-), e.g. 3-phytases (EC 3.1.3.8) and 6-phytases (EC 3.1.3.26); glycosidases (EC 3.2, which fall within a group denoted herein as “carbohydrases”), such as a-amylases (EC 3.2.1.1); peptidases (EC 3.4, also known as proteases); and other carbonyl hydrolases. Examples of commercially available phytases include Bio-Feed® Phytase (Novozymes), Ronozyme® HiPhos (DSM Nutritional Products), Ronozyme™ P (DSM Nutritional Products), NatuphosTM (BASF), FinaseTM (AB Enzymes), and the Phyzyme™ product series (Danisco). Other preferred phytases include those described in WO 98/28408, WO 00/43503, and WO 03/066847.

In the present context, the term “carbohydrase” is used to denote not only enzymes capable of breaking down carbohydrate chains (e.g. starches or cellulose) of especially five- and six-membered ring structures (i.e. glycosidases, EC 3.2), but also enzymes capable of isomerizing carbohydrates, e.g. six-membered ring structures such as D-glucose to five-membered ring structures such as D-fructose.

Carbohydrases of relevance include the following (EC numbers in parentheses): a-amylases (EC 3.2.1.1), 8-amylases (EC 3.2.1.2), glucan 1,4-a-glucosidases (EC 3.2.1.3), endo-1,4-beta-glucanase (cellulases, EC 3.2.1.4), endo-1,3(4)-8-glucanases (EC 3.2.1.6), endo-1,4-8-4anases (EC 3.2.1.8), dextranases (EC 3.2.1.11), chitinases (EC 3.2.1.14), polygalacturonases (EC 3.2.1.15), lysozymes (EC 3.2.1.17), 8-glucosidases (EC 3.2.1.21), ocgalactosidases (EC 3.2.1.22), 8-galactosidases (EC 3.2.1.23), amylo-1,6-glucosidases (EC 3.2.1.33), xylan 1,4-8-4osidases (EC 3.2.1.37), glucan endo-1,3-8-D-glucosidases (EC 3.2.1.39), a-dextrin endo-1,6-oc-glucosidases (EC3.2.1.41), sucrose oc-glucosidases (EC 3.2.1.48), glucan endo-1,3-oc-glucosidases (EC 3.2.1.59), glucan 1,4-8-glucosidases (EC 3.2.1.74), glucan endo-1,6-8-glucosidases (EC 3.2.1.75), galactanases (EC 3.2.1.89), arabinan endo-1,5-a-L-arabinosidases (EC 3.2.1.99), lactases (EC 3.2.1.108), chitosanases (EC 3.2.1.132) and xylose isomerases (EC 5.3.1.5).

In the present context a phytase is an enzyme which catalyzes the hydrolysis of phytate (myo-inositol hexakisphosphate) to (1) myo-inositol and/or (2) mono-, di-, tri-, tetra- and/or pentaphosphates thereof and (3) inorganic phosphate.

According to the ENZYME site referred to above, different types of phytases are known: A so-called 3-phytase (myo-inositol hexaphosphate 3-phosphohydrolase, EC 3.1.3.8) and a so-called 6-phytase (myo-inositol hexaphosphate 6-phosphohydrolase, EC 3.1.3.26). For the purposes of the present invention, both types are included in the definition of phytase.

For the purposes of the present invention phytase activity may be, preferably is, determined in the unit of FYT, one FYT being the amount of enzyme that liberates 1 micro-mol inorganic or-tho-phosphate per min. under the following conditions: pH 5.5; temperature 37° C.; substrate: sodium phytate (C₆H₆O₂₄P₆Na₁₂) in a concentration of 0.0050 mol/I. Suitable phytase assays are described in Example 1 of WO 00/20569. FTU is for determining phytase activity in feed and premix. In the alternative, the same extraction principles as described in Example 1, e.g. for endoglucanase and xylanase measurements, can be used for determining phytase activity in feed and premix. Examples of phytases are disclosed in WO 99/49022 (Phytase variants), WO 99/48380, WO 00/43503 (Consensus phytases), EP 0897010 (Modified phytases), EP 0897985 (Consensus phytases).

In a particular aspect of the present invention the enzyme is selected from the group consisting of endoglucanases, endo-1,3(4)-beta-glucanases, proteases, phytases, galactanases, mannanases, dextranases and alpha-galactosidase, and reference is made to WO 2003/062409 which is hereby incorporated by reference.

Particular suitable feed enzymes include: amylases, phosphotases, such as phytases, and/or acid phosphatases; carbohydrases, such as amylytic enzymes and/or plant cell wall degrading enzymes including cellulases such as β-glucanases and/or hemicellulases such as xylanases or galactanases; proteases or peptidases such as lysozyme; galatosidases, pectinases, esterases, lipases, in particular phospholipases such as the mammalian pancreatic phospholipases A2 and glucose oxidase. In particular the feed enzymes have a neutral and/or acidic pH optimum. In a particular aspect of the present invention the enzyme is selected from the group consisting of amylases, proteases, muramidases, beta-glucanases, phytases, xylanases, phospholipases and glucose oxidases.

Preparation of the Granule

The core of the granule can be prepared by granulating a blend of the ingredients, e.g. by a method comprising granulation techniques such as crystallization, precipitation, pan-coating, fluid bed coating, fluid bed agglomeration, rotary atomization, extrusion, prilling, spheronization, size reduction methods, drum granulation, roller compaction and/or high shear granulation.

Methods for preparing the core of the granule can be found in Handbook of Powder Technology; Particle size enlargement by C. E. Capes; Volume 1; 1980; Elsevier.

Preparation methods include known feed and granule formulation technologies, e.g.:

a) Spray dried products, wherein a liquid enzyme-containing solution is atomized in a spray drying tower to form small droplets which during their way down the drying tower dry to form an enzyme-containing particulate material. Very small particles can be produced this way (Michael S. Showell (editor); Powdered detergents; Surfactant Science Series; 1998; vol. 71; page 140-142; Marcel Dekker).

b) Layered products, wherein the enzyme is coated as a layer around a pre-formed core particle, wherein an enzyme-containing solution is atomized, typically in a fluid bed apparatus wherein the pre-formed core particles are fluidized, and the enzyme-containing solution adheres to the core particles and dries up to leave a layer of dry enzyme on the surface of the core particle. Particles of a desired size can be obtained this way if a useful core particle of the desired size can be found. This type of product is described in e.g. WO 97/23606

c) Absorbed core particles, wherein rather than coating the enzyme as a layer around the core, the enzyme is absorbed onto and/or into the surface of the core. Such a process is described in WO 97/39116.

d) Extrusion or pelletized products, wherein an enzyme-containing paste is pressed to pellets or under pressure is extruded through a small opening and cut into particles which are subsequently dried. Such particles usually have a considerable size because of the material in which the extrusion opening is made (usually a plate with bore holes) sets a limit on the allowable pressure drop over the extrusion opening. Also, very high extrusion pressures when using a small opening increase heat generation in the enzyme paste, which is harmful to the enzyme. (Michael S. Showell (editor); Powdered detergents; Surfactant Science Series; 1998; vol. 71; page 140-142; Marcel Dekker)

e) Prilled products, wherein an enzyme-containing powder is suspended in molten wax and the suspension is sprayed, e.g. through a rotating disk atomiser, into a cooling chamber where the droplets quickly solidify (Michael S. Showell (editor); Powdered detergents; Surfactant Science Series; 1998; vol. 71; page 140-142; Marcel Dekker). The product obtained is one wherein the enzyme is uniformly distributed throughout an inert material instead of being concentrated on its surface. Also U.S. Pat. Nos. 4,016,040 and 4,713,245 are documents relating to this technique

f) Mixer granulation products, wherein an enzyme is added in dry form together with a liquid or in liquid form to a dry powder composition of conventional granulating components. The liquid and the powder in a suitable proportion are mixed and as the moisture of the liquid is absorbed in the dry powder, the components of the dry powder will start to adhere and agglomerate and particles will build up, forming granules comprising the enzyme. Such a process is described in U.S. Pat. No. 4,106,991 and related documents EP 170360, EP 304332, EP 304331, WO 90/09440 and WO 90/09428. In a particular product of this process wherein various high-shear mixers can be used as granulators, granules consisting of enzyme, fillers and binders etc. are mixed with cellulose fibers using melt granulation to reinforce the particles to give the so-called T-granule. Reinforced particles, being more robust, release less enzymatic dust.

g) Size reduction, wherein the cores are produced by milling or crushing of larger particles, pellets, tablets, briquettes etc. containing the enzyme. The wanted core particle fraction is obtained by sieving the milled or crushed product. Over and undersized particles can be recycled. Size reduction is described in (Martin Rhodes (editor); Principles of Powder Technology; 1990; Chapter 10; John Wiley & Sons). The initial larger particles can be obtained by methods such as roller compaction of a powder.

h) Fluid bed granulation. Fluid bed granulation involves suspending particulates in an air stream and spraying a liquid onto the fluidized particles via nozzles. Particles hit by spray droplets get wetted and become tacky.

i) The cores may be subjected to drying, such as in a fluid bed drier. Other known methods for drying granules in the feed or enzyme industry can be used by the skilled person. The drying preferably takes place at a product temperature of from 25 to 90° C. For some enzymes it is important the cores comprising the enzyme contain a low amount of water before coating with the salt. If water sensitive enzymes are coated with a salt before excessive water is removed, it will be trapped within the core and it may affect the activity of the enzyme negatively. After drying, the cores preferably contain 0.1-10% w/w water.

The granule may optionally be surrounded by at least one coating in addition to the coating described above, e.g. to improve the storage stability or to reduce the dust formation. The optional coating(s) may include a salt coating and/or another type of coating described below.

Optional Salt Coating

The optional salt coating may comprise up to 30% by weight w/w of the granule.

The coating may be applied in an amount of at least 1% by weight of the core, e.g. at least 3% or 5%. The amount may be at most 30%, such as at the most 20%, 15% or 10% by weight of the core.

To provide acceptable protection, the salt coating is preferably at least 1 μm thick. In a particular aspect the thickness of the salt coating is below 25 μm. In a more particular aspect the thickness of the salt coating is below 20 μm. In an even more particular aspect the total thickness of the salt coating is below 15 μm.

The coating should encapsulate the core unit by forming a substantially continuous layer. A substantially continuous layer is to be understood as a coating having few or no holes, so that the core unit it is encapsulating/enclosing has few or none uncoated areas. The layer or coating should in particular be homogeneous in thickness. The salt may be added from a salt solution where the salt is completely dissolved or from a salt suspension wherein the fine particles is less than 50 μm, such as less than 10 μm.

The salt coating can further contain other materials as known in the art, e.g. fillers, antisticking agents, pigments, dyes, plasticizers and/or binders, such as titanium dioxide, kaolin, calcium carbonate or talc.

Salts

The salt coating may comprise a single salt or a mixture of two or more salts. The salt may be water soluble, in particular having a solubility at least 0.1 grams in 100 g of water at 20° C., preferably at least 0.5 g per 100 g water, e.g. at least 1 g per 100 g water, e.g. at least 5 g per 100 g water.

The salt may be an inorganic salt, e.g. salts of sulfate, sulfite, phosphate, phosphonate, nitrate, chloride or carbonate or salts of simple organic acids (less than 10 carbon atoms e.g. 6 or less carbon atoms) such as citrate, malonate or acetate. Examples of cations in these salts are alkali or earth alkali metal ions, the ammonium ion or metal ions of the first transition series, such as sodium, potassium, magnesium, calcium, zinc or aluminium. Examples of anions include chloride, bromide, iodide, sulfate, sulfite, bisulfite, thiosulfate, phosphate, monobasic phosphate, dibasic phosphate, hypophosphite, dihydrogen pyrophosphate, tetraborate, borate, carbonate, bicarbonate, metasilicate, citrate, malate, maleate, malonate, succinate, lactate, formate, acetate, butyrate, propionate, benzoate, sorbate, tartrate, ascorbate or gluconate. In particular alkali- or earth alkali metal salts of sulfate, sulfite, phosphate, phosphonate, nitrate, chloride or carbonate or salts of simple organic acids such as citrate, malonate or acetate may be used.

The salt in the coating may have a constant humidity at 20° C. above 60%, particularly above 70%, above 80% or above 85%, or it may be another hydrate form of such a salt (e.g. anhydrate). The salt coating may be as described in WO 00/01793 or WO 2006/034710.

Specific examples of suitable salts are NaCl (CH_{20° C.}=76%), Na₂CO₃ (CH_{20° C.}=92%), NaNO₃ (CH_{20° C.}=73%), Na₂HPO₄ (CH_{20° C.}=95%), Na₃PO₄ (CH_{25° C.}=92%), NH₄Cl (CH_{20° C.}=79.5%), (NH₄)₂HPO₄. (CH_{20° C.}=93.0%), NH₄H₂PO₄ (CH_{20° C.}=93.1%), (NH₄)₂SO₄ (CH_{20° C.}=81.1%), KCl (CH_{20° C.}=85%), K₂HPO₄ (CH_{20° C.}=92%), KH₂PO₄ (CH_{20° C.}=96.5%), KNO₃ (CH_{20° C.}=93.5%), Na₂SO₄ (CH_{20° C.}=93%), K₂SO₄ (CH_{20° C.}=98%), KHSO₄ (CH_{20° C.}=86%), MgSO₄ (CH_{20° C.}=90%), ZnSO₄ (CH_{20° C.}=90%) and sodium citrate (CH_{25° C.}=86%). Other examples include NaH₂PO₄, (NH₄)H₂PO₄, CuSO₄, Mg(NO₃)₂ and magnesium acetate.

The salt may be in anhydrous form, or it may be a hydrated salt, i.e. a crystalline salt hydrate with bound water(s) of crystallization, such as described in WO 99/32595. Specific examples include anhydrous sodium sulfate (Na₂SO₄), anhydrous magnesium sulfate (MgSO₄), magnesium sulfate heptahydrate (MgSO₄ 7H₂O), zinc sulfate heptahydrate (ZnSO₄ 7H₂O), sodium phosphate dibasic heptahydrate (Na₂HPO₄ 7H₂O), magnesium nitrate hexahydrate (Mg(NO₃)₂(6H₂O)), sodium citrate dihydrate and magnesium acetate tetrahydrate. Preferably the salt it applied as a solution of the salt e.g. using a fluid bed.

Optional Additional Coating

The granule may optionally have one or more additional coatings. Examples of suitable coating materials are polyethylene glycol (PEG), methyl hydroxy-propyl cellulose (MHPC) and polyvinyl alcohol (PVA).

### Preferred Embodiments

The invention is further described by the following preferred embodiments:

1. Use of a low dust enzyme granule for post pelleting liquid application (PPLA) or liquid application on other types of non-pelleted feed, such as mash feed of at least one enzyme, wherein the enzyme granule is dissolved in water before application.

2. Use of an enzyme granule for post pelleting liquid application (PPLA) or liquid application on other types of non-pelleted feed, such as mash feed of at least one enzyme, wherein the enzyme granule has a low tendency of dust formation, and wherein the enzyme granule is dissolved in water before application.

3. The use according to embodiment 1 or 2, wherein the dissolved granule is applied onto pellets or mash feed as a liquid composition by spray.

4. The use according to embodiment 3, wherein the dissolved granule is applied onto pellets.

5. The use according to embodiment 4, wherein the dissolved granule is applied onto heat treated pellets.

6. The use according to any one of embodiments 1 to 5, wherein the dissolved granule is sprayed onto pellets or mash feed as a liquid composition by spraying at the die, spraying into a screw conveyor, spraying into a plenum or weir or spraying using spinning disks to atomize the liquid.

7. The use according to embodiment 6, wherein the granule is dissolved and sprayed onto feed pellets or mash feed in a feed mill.

8. The use according to any one of embodiments 1 to 7, wherein an acid is dissolved in water together with the granule before application.

9. The use according to embodiment 8, where the acid is selected from the group consisting of: sorbic acid, ascorbic acid, citric acid, benzoic acid and mixtures thereof.

10. The use according to embodiment 9, where the acid is citric acid.

11. The use according to any one of embodiments 1 to 10, wherein the enzyme granule results in little or no total dust release during handling.

12. The use according to any one of embodiments 1 to 11, where the total dust is below 1000 pg/g when measured in the Heubach Type 1 assay and/or below 1000 μg/g when measured in Elutriation assay.

13. The use according to any one of embodiments 1 to 12, wherein total dust is below 500 pg/g when measured in Heubach Type 1 assay, below 250 μg/g in Heubach Type 1 assay, below 100 μg/g in Heubach Type 1 assay or below 50 μg/g in Heubach Type 1 assay.

14. The use according to any one of embodiments 1 to 13, wherein total dust is below 500 pg/g when measured in Elutriation assay, below 250 μg/g in Elutriation assay, below 100 μg/g in Elutriation assay or below 50 μg/g in Elutriation assay.

15. The use according to any one of embodiments 1 to 14, wherein active dust fraction is below 20 ppm when measured in the Heubach Type 1 assay and/or below 80 ppm when measured in Elutriation assay.

16. The use according to any one of embodiments 1 to 15, wherein active dust fraction is below 10 ppm when measured in Heubach Type 1 assay, below 6 ppm, below 2 ppm or below 0.5 ppm in Heubach Type 1 assay.

17. The use according to any one of embodiments 1 to 16, wherein active dust fraction is below 40 ppm when measured in Elutriation assay, below 20 ppm, below 10 ppm, below 4 ppm, below 2 ppm, or below 0.5 ppm in Elutriation assay.

18. The use according to any one of embodiments 1 to 17, wherein the granule is a mixer granulation product, a compacted powder granule, a prilled granule, extrudated granule, or a layered granule.

19. The use according to embodiment 18, wherein the granule is a layered granule.

20. The use according to any one of embodiments 1 to 19, wherein the granule comprises a core and one or more enzyme-comprising layers, wherein the enzyme-comprising layer comprises an enzyme and a binder e.g. a carbohydrate.

21. The use according to any one of embodiments 1 to 20, wherein the granule has an outer coating over the enzyme-comprising layer.

22. The use according to any one of embodiments 1 to 21, wherein the granule further comprises a microbial stabilizer.

23. The use according to embodiment 22, wherein the microbial stabilizer in the granule is present in the core, the enzyme-containing layer, the outer coating, or any combination thereof.

24. The use according to any one of embodiments 22 to 23, wherein the microbial stabilizer is present in an amount between 5-50% of the granule.

25. The use according to any one of embodiments 22 to 24, wherein the microbial stabilizer is present in an amount between 20-40% of the granule.

26. The use according to any one of embodiments 1 to 25, wherein the granule comprises a core and one or more enzyme-comprising layers, wherein the core comprises an inert material and/or a microbial stabilizer and the enzyme-comprising layer comprises an enzyme and a binder e.g. a carbohydrate.

27. The use according to embodiment 26, wherein the granule comprises one enzyme-comprising layer which is coated over the core and the enzyme-comprising layer comprises an enzyme and a binder e.g. a carbohydrate.

28. The use according to any one of embodiments 1 to 27, wherein the granule comprises a core and an enzyme-comprising layer coated over the core, wherein the core comprises an inert material and the enzyme-comprising layer comprises an enzyme and a binder e.g. a carbohydrate.

29. The use according to any one of embodiments 1 to 28, wherein the granule comprises a core and an enzyme-comprising layer coated over the core, wherein the core comprises a microbial stabilizer and the enzyme-comprising layer comprises an enzyme and a binder e.g. a carbohydrate.

30. The use according to any one of embodiments 20 to 29, wherein the binder is a carbohydrate.

31. The use according to any one of embodiments 20 to 30, wherein the carbohydrate is selected from the group consisting of: fructose, sucrose, maltose, dextrin, maltodextrin, galactose, mannose, mannitol, glucose, lactose and sorbitol.

32. The use according to any one of embodiments 20 to 31, wherein the carbohydrate is selected from the group consisting of: dextrin and sucrose.

33. The use according to any one of embodiments 20 to 32, wherein the carbohydrate is dextrin.

34. The use according to any one of embodiments 20 to 32, wherein the carbohydrate is sucrose.

35. The use according to any one of embodiments 20 to 34, wherein the ratio between the carbohydrate and the active enzyme in the granule is such that the carbohydrate is present in the granule in an amount of between 30% to 130% of the active enzyme amount when measured as dry solids.

36. The use according to any one of embodiments 1 to 35, wherein the granule comprises a core and an enzyme-comprising layer coated over the core, wherein the core comprises an inert material and the enzyme-comprising layer comprises an enzyme and dextrin.

37. The use according to any one of embodiments 20 to 36, wherein the core comprises further ingredients selected from the group consisting of: binders, active ingredients, enzyme stabilizers, microbial stabilizers and combinations thereof.

38. The use according to any one of embodiments 20 to 37, wherein the core comprises an inert material which is selected from the group consisting of: Sodium sulfate, sodium chloride, sodium carbonate, sodium nitrate, sodium phosphate, sodium hydrogen phosphate, ammonium sulfate, ammonium chloride, ammonium carbonate, ammonium nitrate, ammonium phosphate, ammonium hydrogen phosphate, potassium sulfate, potassium chloride, potassium carbonate, potassium nitrate, potassium phosphate, potassium hydrogen phosphate, magnesium sulfate, zinc sulfate, sodium citrate, a sugar, a carbohydrate (such as e.g. sucrose, dextrin, glucose, lactose or sorbitol) and combinations thereof.

39. The use according to any one of embodiments 20 to 38, wherein the core comprises an inert material which is selected from the group consisting of: Sodium sulfate, sodium chloride and a mixture thereof.

40. The use according to any one of embodiments 20 to 39, wherein the core comprises a microbial stabilizer which is selected from the group consisting of: sorbic acid, ascorbic acid, citric acid, benzoic acid, a salt of sorbic acid, a salt of ascorbic acid, a salt of citric acid, a salt of benzoic acid, potassium sorbate, sodium citrate, sodium benzoate and combinations thereof.

41. The use according to any one of embodiments 20 to 40, wherein the microbial stabilizer in the core is selected from the group consisting of: sorbic acid and a salt thereof, ascorbic acid and a salt thereof, citric acid and a salt thereof, benzoic acid and a salt thereof, potassium sorbate, sodium citrate and/or sodium benzoate and combinations thereof.

42. The use according to embodiment 40 or 41, where the salt of sorbic acid is sodium sorbate or potassium sorbate, the salt of ascorbic acid is sodium ascorbate or potassium ascorbate, the salt of citric acid is sodium citrate or potassium citrate, and/or the salt of benzoic acid is sodium benzoate or potassium benzoate.

43. The use according to any one of embodiments 20 to 42, wherein the microbial stabilizer in the core is selected from: benzoic acid, sorbic acid, a salt of benzoic acid, a salt of sorbic acid and combinations thereof.

44. The use according to any one of embodiments 20 to 43, wherein the microbial stabilizer in the core is sodium benzoate or potassium benzoate.

45. The use according to any one of embodiments 20 to 44, wherein the microbial stabilizer in the core further comprises a weak acid such as benzoic acid, citric acid, sorbic acid or acetic acid.

46. The use according to any one of embodiments 20 to 45, wherein the microbial stabilizer is a mixture of sorbic acid and potassium sorbate.

47. The use according to any one of embodiments 20 to 46, wherein the microbial stabilizer in the core is sorbic acid and wherein potassium sorbate is added to the enzyme-layer.

48. The use according to any one of embodiments 21 to 47, where the outer coating comprises a salt and optionally one or more organic coating materials such as waxes (e.g. polyethylene glycols, polypropylenes, Carnauba wax, Candelilla wax, bees wax, hydrogenated plant oil or animal tallow, hydrogenated palm oil, fatty acid alcohols, mono-glycerides and/or di-glycerides, micro-crystalline wax, paraffins, and/or fatty acids).

49. The use according to any one of embodiments 21 to 48, where the outer coating further comprises a microbial stabilizer.

50. The use according to embodiment 49, wherein the microbial stabilizer in the outer coating is selected from the group consisting of: sorbic acid, ascorbic acid, citric acid, benzoic acid, a salt of sorbic acid, a salt of ascorbic acid, a salt of citric acid, a salt of benzoic acid, potassium sorbate, sodium citrate, sodium benzoate and combinations thereof.

51. The use according to any one of embodiments 49 to 50, wherein the microbial stabilizer in the outer coating is selected from the group consisting of: sorbic acid and a salt thereof, ascorbic acid and a salt thereof, citric acid and a salt thereof, benzoic acid and a salt thereof, potassium sorbate, sodium citrate, sodium benzoate and combinations thereof.

52. The use according to embodiment 51, where the salt of sorbic acid is sodium sorbate or potassium sorbate, the salt of ascorbic acid is sodium ascorbate or potassium ascorbate, the salt of citric acid is sodium citrate or potassium citrate, and/or the salt of benzoic acid is sodium benzoate or potassium benzoate.

53. The use according to any one of embodiments 49 to 52, wherein the microbial stabilizer in the outer coating is selected from the group consisting of: sorbic acid, ascorbic acid, citric acid, benzoic acid, potassium sorbate, sodium citrate, sodium benzoate and combinations thereof.

54. The use according to any one of embodiments 49 to 53, wherein the microbial stabilizer in the outer coating is ascorbic acid and/or citric acid.

55. The use according to any one of embodiments 49 to 53, wherein the microbial stabilizer in the outer coating is a mixture of sorbic acid and potassium sorbate.

56. The use according to any one of embodiments 48 to 55, wherein the salt in the outer coating is selected from the group consisting of: sodium sulfate, sodium chloride, sodium carbonate, sodium nitrate, sodium phosphate, sodium hydrogen phosphate, ammonium sulfate, ammonium chloride, ammonium carbonate, ammonium nitrate, ammonium phosphate, ammonium hydrogen phosphate, potassium sulfate, potassium chloride, potassium carbonate, potassium nitrate, potassium phosphate, potassium hydrogen phosphate, magnesium sulfate, zinc sulfate, sodium citrate, potassium sorbate, sodium benzoate, sodium ascorbate and mixtures thereof.

57. The use according to any one of embodiments 48 to 56, wherein the salt in the outer coating is selected from the group consisting of: sodium citrate, potassium sorbate, sodium benzoate and sodium ascorbate.

58. The use according to embodiment 56, wherein the salt in the outer coating is sodium sulfate.

59. The use according to any one of embodiments 20 to 58, wherein microbial stabilizers are present in the core, the enzyme-containing layer and the outer coating of the granule.

60. The use according to embodiment 59, wherein sorbic acid is present in the core, potassium sorbate is present in the enzyme-layer, and sodium sulfate is present in the outer coating.

61. The use according to any one of embodiments 1 to 60, wherein the granule comprises at least 10% active enzyme such as at least 20% active enzyme or at least 30% active enzyme.

62. The use according to one of embodiments 1 to 61, wherein the enzyme is selected from the group consisting of amylase, protease, beta-glucanase, phytase, muramidase, xylanase, phospholipase and glycose oxidase or a mixture thereof.

63. The use according to any one of embodiments 1 to 62, wherein the granule is a layered granule produced in a fluid bed process.

64. The use according to embodiment 63, where the granule has a characteristic onion type structure.

65. The use according to embodiment 63 or 64, where the granule has concentric uniform layers.

66. The use according to any one of embodiments 1 to 65, wherein the particle size distribution (PSD) of the granule has a D50 of at least 400 μm, and a D10 of at least 300 μm.

67. The use according to embodiment 66, wherein PSD of the granule has a D90 of up to 1400 μm.

68. The use according to embodiment 67, wherein PSD of the granule has a D90 of up to 1200 μm.

69. The use according to any one of embodiments 66 to 68, wherein PSD of the granule has a D50 of between 500 and 1000 μm.

70. The use according to any one of embodiments 1 to 69, wherein the concentration of the granule is between 0.5 to 25% when it is dissolved in water before application.

71. The use according to embodiment 70, wherein the concentration of the granule is between 0.5 to 10%.

72. The use according to embodiment 71, wherein the concentration of the granule is between 0.5 to 5%.

73. The use according to any one of embodiments 1 to 72, wherein pH is between 3.5 to 5.5 when the granule is dissolved in water in a concentration of 1.5%.

74. The use according to any one of embodiments 1 to 73, wherein the granule is soluble in water.

75. The use according to embodiment 74, wherein the solubility of the granule is determined by a) dissolving the granule at 1.5% concentration in water, b) sieving the solution from step a) through a 100 micrometer sieve, c) drying the sieve and d) checking the weight of insoluble matter captured by the sieve;

wherein the granule is soluble in water if there is less than 0.5% residual matter.

76. The use according to embodiment 75, wherein the granule is soluble in water if there is less than 0.25% residual matter.

77. The use according to embodiment 75, wherein the granule is soluble in water if there is less than 0.1% residual matter.

78. Process for producing a low dust enzyme granule for the use according to any one of embodiments 1 to 77, comprising preparing a granule comprising a core and at least one enzyme wherein the enzyme is distributed in the core and/or layered over the core, and applying to the core or layered granule an outer layer to obtain a coated granule.

79. The process according to embodiment 78, wherein the granule is prepared in a fluid bed apparatus.

### EXAMPLES

Analytical Methods

Total dust determined by Heubach Type 1

Total dust (dust from the active and the non-active granule ingredients) was determined by the well-known method Heubach Type 1. In the assay, the weighed-out sample amount was placed in a rotating drum containing three integrated blades. A horizontal air stream passed through the drum with a flow at 20 L/min. The airflow led the finest particles further through a non-rotating, horizontal glass column in which the largest particles were separated. The airborne dust was led further and collected on a filter in the filter house. The amount of enzyme dust on the filter was determined by weighing the filter house before and after analysis. The result is expressed as μg of dust released per g of product.

Conditions of Analysis:

Temperature: Room temperature

Sample amount: 50.0 g

Air flow: 20 L/min.

Speed of rotation: 30 rpm

Time of analysis: 5 min.

Humidity of air: 30-70% RH

Fiber glass filter: 5 cm GF92

Active Dust Fraction Determined by Heubach Type 1 Dustmeter

The amount of active enzyme on the filter (obtained from the Heubach Type 1 method as explained in the total dust determination) was determined by means of an analytical method for dust filters for the enzyme in question. The activity of the enzyme on the dust filter was determined and the active dust fraction was obtained by dividing the activity of the enzyme on the dust filter released per gram of sample, by the total activity of the enzyme per gram of sample, and was expressed as ppm (activity obtained on dust filter/total activity on product ×10⁶).

Total Dust Determined by Elutriation

In the assay, the enzyme granule was fluidized using air in a glass column. The released dust was collected on a glass fiber filter. The amount of enzyme dust on the filter was determined by weighing the filter before and after analysis. The result is expressed as μg of dust released per g of product.

Conditions of Analysis:

Temperature: Room temperature

Sample amount: 60.0 g

Air flow: 2.83 m³/hour ˜0.8 m/s

Time of analysis: 40 min.

Humidity of air: 0-1% RH

Fiber glass filter: Ø15 cm Whatman GF/C CAT no. 1822-150

Active dust fraction determined by Elutriation

The amount of active enzyme on the filter (obtained from the Elutriation method as explained in the total dust determination) was determined by means of an analytical method for dust filters for the enzyme in question. The activity of the enzyme on the dust filter was determined and the active dust fraction was obtained by dividing the activity of the enzyme on the dust filter released per gram of sample, by the total activity of the enzyme per gram of sample, and is expressed as ppm (activity obtained on dust filter/total activity on product ×10⁶).

Determination of Active Enzyme Content

Active enzyme content was determined using the relevant enzyme activity method. A correlation between activity and enzyme content (amounts in e.g. g/kg material) can be determined by activity measurements and protein concentration determination (e.g. SDS-PAGE, amino-acid analysis, purification from product and quantification). Active enzyme content is calculated dividing activity per gram of product by the specific activity of the enzyme (activity released per gram of pure enzyme) and is expressed in weight %.

As example, phytase activity was determined by the well-known FYT method. Other recognized methods by the ISO 30024:2009 could be used, such as FTU or OTU. The following is an example on how to determine phytase activity on a micro-titer plate setup:

75 microliter phytase-containing enzyme solution, appropriately diluted in 0.25M sodium acetate, 0.005% (w/v) Tween-20. pH5.5, is dispensed in a microtiter plate well, e. g. NUNC 269620, and 75 microliter substrate is added (prepared by dissolving 100mg sodium phytate from rice (Aldrich Cat.No. 274321) in 10 ml 0.25M sodium acetate buffer, pH5.5). The plate is sealed and incubated 15min. shaken with 750rpm at 37° C. After incubation, 75 microliter stop reagent is added (the stop reagent being prepared by mixing 10 ml molybdate solution (10% (w/v) ammonium hepta-molybdate in 0.25% (w/v) ammonia solution), 10 ml ammonium vanadate (0.24% commercial product from Bie&Berntsen, Cat.No. LAB17650), and 20 ml 21.7% (w/v) nitric acid), and the absorbance at 405 nm is measured in a microtiter plate spectrophotometer. The phytase activity is expressed in the unit of FYT, one FYT being the amount of enzyme that liberates 1 micromole inorganic ortho-phosphate per minute under the conditions above. An absolute value for the measured phytase activity may be obtained by reference to a standard curve prepared from appropriate dilutions of inorganic phosphate, or by reference to a standard curve made from dilutions of a phytase enzyme preparation with known activity (such standard enzyme preparation with a known activity is available on request from Novozymes A/S, Krogshoejvej 36, DK-2880 Bagsvaerd).

Flowability Assessment

Flowability of a granule or powder sample can be determined in different ways. One typical method is by evaluating the so-called “angle of repose” where the steepest angle of descent relative to the horizontal plane to which a material can be piled without slumping is measured. At this angle, the material on the slope face is on the verge of sliding. The angle of repose can range from 0° to 90°.

### EXAMPLE DESCRIPTIONS

The products in below examples were produced by a layering granulation process, where a core was covered by a series of layers containing the active ingredients in the product.

### Example 1

### Enzyme Layer on Salt Core

Example 1 covers the product in its simplest form. The core material was a fast dissolving salt and the enzyme was applied in a single layer.

Na₂SO₄ cores, PSD 250-355 μm, were prepared by sieving in a Russel-Finex C400 2500 g cores were loaded into a Glatt Procell GF3 fluid bed

A feed was prepared:

12300 g Phytase concentrate (Ronozyme® HiPhos), purified by UF and concentrated to 39.5% DS

632 g dextrin Avedex W80.

The feed was sprayed onto the cores in the fluid bed applying the following configuration and process parameters:

Mode of operation: Bottom spray

Nozzle: 1,2 mm

Nozzle air pressure: 2,4 bar

Air flow: 140-150 m³/hour

Air temperature: 100° C.

Feed flow: 45-55 g/min

Product temp. coating: 50-55° C.

Product temp. drying: 60° C.

### Example 2

### Salt Layer Coated Product

A product produced according to example 1 was given a second layer coat. In the simplest form the second layer was made of a fast dissolving salt. The salt was applied under process conditions for high uniformity of the layer.

Na₂SO₄ cores, PSD 250-355 μm, were prepared by sieving in a Russel-Finex C400 2500 g cores were loaded into a Glatt Procell GF3 fluid bed

A feed for the enzyme layer was prepared:

13100 g Phytase concentrate (Ronozyme® HiPhos), purified by UF and concentrated to 39.5% DS

326 g dextrin Avedex W80.

The feed was sprayed onto the cores in the fluid bed applying the following configuration and process parameters:

Mode of operation: Bottom spray

Nozzle: 1,2 mm

Nozzle air pressure: 2,4 bar

Air flow: 90-130 m³/hour

Air temperature: 100° C.

Feed flow: 45-65 g/min

Product temp. coating: 55-63° C.

Product temp. drying: 60° C.

4000 g of this product was reloaded into the Glatt Procell GF3 fluid bed

A feed for the salt layer was produced: 348 g Na₂SO₄

852 g water at 40-45° C.

The feed was sprayed onto the product in the fluid bed applying the following configuration and process parameters:

Mode of operation: Bottom spray

Nozzle: 1,2 mm

Nozzle air pressure: 1,2-1,8 bar

Air flow: 140-150 m³/hour

Air temperature: 130° C.

Feed flow: 90-145 g/min

Product temp. coating: 50-55° C.

Product temp. drying: 60° C.

### Example 3

### Wax Layer Coated Product

A product produced according to example 2 was given a third layer. The material for this layer was a wax, a polymer or an oil or a mix thereof.

2500 g of the product produced in example 1 was reloaded into the Glatt Procell GF3 fluid bed.

A feed for a thicker enzyme layer was prepared:

12300 g Phytase concentrate (Ronozyme® HiPhos), purified by UF and concentrated to 39.5% DS

632 g dextrin Avedex W80.

The feed was sprayed onto the product in the fluid bed applying the same process parameters as for example 1.

7000 g of this product was reloaded into the Glatt Procell GF3 fluid bed.

A feed for the salt layer was prepared:

413 g Na₂SO₄

1011 g water at 40-45° C.

The feed was sprayed onto the product in the fluid bed applying the following configuration and process parameters:

Mode of operation: Bottom spray

Nozzle: 1,2 mm

Nozzle air pressure: 3,0-3,5 bar

Air flow: 160 m³/hour

Air temperature: 170° C.

Feed flow: 110-200 g/min

Product temp. coating: 50-55° C.

Product temp. drying: 60° C.

4000 g of the salt layer coated product was reloaded into the Glatt Procell GF3 fluid bed.

A feed for the wax layer was prepared:

68 g PEG 4000

100 g HPMC

1103 g water

The feed was sprayed onto the product in the fluid bed applying the following configuration and process parameters:

Mode of operation: Bottom spray

Nozzle: 1,2 mm

Nozzle air pressure: 2,0 bar

Air flow: 150 m³/hour

Air temperature: 80° C.

Feed flow: 20-30 g/min

Product temp. coating: 45° C.

### Example 4

### Product with Microbial Stabilizer

In this product, the microbial stabilizer sodium benzoate was incorporated into the layer granulation process as the material for the cores.

Sodium benzoate cores, PSD 250-500 μm, were prepared by sieving in a Russel-Finex C400 2000 g cores were loaded into a Glatt Procell GF3 fluid bed.

The enzyme layer was applied in two steps as in example 3.

The feed for the first enzyme layer was produced: 13480 g Phytase concentrate (Ronozyme® HiPhos), purified by UF and concentrated to 39.5% DS

692 g dextrin Avedex W80.

This feed was sprayed onto the cores in the fluid bed applying the following configuration and process parameters:

Mode of operation: Bottom spray

Nozzle: 1,2 mm

Nozzle air pressure: 1,2-2,0 bar

Air flow: 70-150 m³/hour

Air temperature: 100° C.

Feed flow: 10-65 g/min

Product temp. coating: 55° C.

Product temp. drying: 60° C.

2500 g of this product was reloaded into the Glatt Procell GF3 fluid bed.

The feed for the second enzyme layer was produced:

12300 g Phytase concentrate (Ronozyme® HiPhos), purified by UF and concentrated to 39.5% DS

632 g dextrin Avedex W80.

This feed was sprayed onto the product in the fluid bed applying the following configuration and process parameters:

Mode of operation: Bottom spray

Nozzle: 1,2 mm

Nozzle air pressure: 1,5-2,0 bar

Air flow: 90-130 m³/hour

Air temperature: 90° C.

Feed flow: 10-60 g/min

Product temp. coating: 55° C.

Product temp. drying: 60° C.

7000 g of this product was reloaded into the Glatt Procell GF3 fluid bed.

A feed for the salt layer was prepared:

455 g Na₂SO₄

1114 g water at 40-45° C.

The feed was sprayed onto the product in the fluid bed applying the following configuration and process parameters:

Mode of operation: Bottom spray

Nozzle: 1,2 mm

Nozzle air pressure: 1,5-2,4 bar

Air flow: 160 m³/hour

Air temperature: 130° C.

Feed flow: 45-145 g/min

Product temp. coating: 55° C.

Product temp. drying: 60° C.

4000 g of the salt layer coated product was reloaded into the Glatt Procell GF3 fluid bed.

A feed for the wax layer was prepared:

68 g PEG 4000

100 g HPMC

904 g water

The feed was sprayed onto the product in the fluid bed applying the following configuration and process parameters:

Mode of operation: Bottom spray

Nozzle: 1,2 mm

Nozzle air pressure: 2,0 bar

Air flow: 150 m³/hour

Air temperature: 80° C.

Feed flow: 35-60 g/min

Product temp. coating: 45° C.

### Example 5

### pH Controlling Agent in Combination with Microbial Stabilizer

This product included citric acid as a pH controlling agent for completion of the microbial stabilizer system. The citric acid was incorporated in the formulation as a granule with a PSD matching the enzyme granule as a means for control of the product homogeneity.

1000 g product produced according to example 4, except for the final wax coat

131 g citric acid monohydrate

was combined into a tumbling mixer and homogenized for 10 min. The citric acid amount was selected so that the pH of a solution of the granule mixture in water, at a concentration of 1.5% w/w was in the range between 4.0-4.5. At this low pH, and with the resulting concentration of Na-benzoate in water, the solution was microbially stable.

### Example 6

### Products with Separation Layers

The layer of enzyme may be separated from the core and from the outer layers by thin layers of salts, sugars or dextrins. In this example, the enzyme layer was separated from the sodium benzoate in the core for improvement of the stability during production.

Sodium benzoate cores, PSD 250-800 μm, were prepared by sieving in a Russel-Finex C400 2500 g cores were loaded into a Glatt Procell GF3 fluid bed.

A feed for the salt separation layer was produced:

325 g Na₂SO₄

796 g water at 40-45° C.

The feed was sprayed onto the product in the fluid bed applying the following configuration and process parameters:

Mode of operation: Bottom spray

Nozzle: 1,2 mm

Nozzle air pressure: 1,2-1,8 bar

Air flow: 140-150 m³/hour

Air temperature: 130° C.

Feed flow: 90-145 g/min

Product temp. coating: 50-55° C.

A feed for the first enzyme layer was produced:

12131 g Phytase concentrate (Ronozyme® HiPhos), purified by UF and concentrated to 39,5% DS

383 g dextrin Avedex W80.

The feed was sprayed onto the material in the fluid bed applying same conditions as used for the enzyme layer in example 2.

The fluid bed was cleared until 2500 g of product was left, and a second enzyme layer was applied:

4763 g Phytase concentrate (Ronozyme® HiPhos), purified by UF and concentrated to 39,5% DS

150 g dextrin Avedex W80.

Feed for a final salt layer was produced:

315 g Na₂SO₄

771 g water at 40-45° C.

The feed was sprayed onto the second layer material in the fluid bed applying process conditions as for the salt layer in example 2.

The product was formulated with citric acid for pH control in a process as described in example 5:

2000 g product coated with the salt layer

367 g citric acid monohydrate

was combined into a tumbling mixer and homogenized for 10 min.

### Example 7

### Enzyme Stabilizers

Specific stabilizers for the specific enzyme may be added into the enzyme and binder layer. Here zinc acetate is introduced as stabilizer for a phytase.

### Example 8

### Dust, Flowability, Solubility and Activity of the Granules

The granules prepared in examples 1 to 6 and 9 to 15 and a state of the art water soluble powder marketed for PPLA were tested for dust, flowability, solubility and activity of the granules. The test results are provided in table 1.

TABLE 1

| | Active dust | Active dust | Total | | | | |
|---|---|---|---|---|---|---|---|
| | fraction as | fraction as | dust by | Total | | | |
| | measured by | measured by | Heubach | dust by | | | Activev Enzyme |
| | Heubach | Elutriation | Type 1 | Elutriation | | | content in |
| | Type 1 | dust | (μg/g | (μg/g | | Solubility | weight basis |
| | (ppm) | (ppm) | product) | product) | Flowability | (after 5 min) | (%) |
| State of the art | 228 | — | 35744* | — | Requires high | No undissolved | >40% |
| Water Soluble Powder | | | | | mechanical impact | matter | |
| marketed for PPLA | | | | | Segregates readily | | |
| Example 1 | — | 12 | — | 90 | Easy and | No undissolved | 31% |
| | | | | | free flowing | matter | |
| Example 2 | 0.3 | 0.2 | 0 | 27 | Easy and | No undissolved | 30% |
| | | | | | free flowing | matter | |
| Example 3 | — | 0.2 | — | 23 | Easy and | No undissolved | 35% |
| | | | | | free flowing | matter | |
| Example 4 | — | 0.0 | — | 12 | Easy and | No undissolved | 34% |
| | | | | | free flowing | matter | |
| Example 5 | — | — | — | — | Easy and | No undissolved | 28% |
| | | | | | free flowing | matter | |
| Example 6 | — | — | — | — | Easy and | No undissolved | 27% |
| | | | | | free flowing | matter | |
| Example 9 | — | 9 | — | 33 | Easy and | Undissolved | 34% |
| | | | | | free flowing | matter | |
| Example 10 | 0.01 | 0.1 | 10 | 0 | Easy and | No undissolved | 20% |
| | | | | | free flowing | matter | |
| Example 11 | 0.2 | 1 | 0 | 0 | Easy and | No undissolved | 19% |
| | | | | | free flowing | matter | |
| Example 12 | — | 2 | — | 8 | Easy and | No undissolved | 35% |
| | | | | | free flowing | matter | |
| Example 13 | — | 0.2 | — | 0 | Easy and | No undissolved | 25% |
| | | | | | free flowing | matter | |
| Example 14 | 1.1 | 18 | 2 | 37 | Easy and | No undissolved | 23% |
| | | | | | free flowing | matter | |
| Example 15 | 1.9 | 46 | 0 | 87 | Easy and | No undissolved | 24% |
| | | | | | free flowing | matter | |

* The dust measurement has variation, 16256 μg/g was obtained in a different run, where active dust measurement was not performed.

### Example 9

### Fully Integrated Coformulation of Enzyme and Microbial Stabilizer

In this example the full microbial stabilizer system was integrated in the individual granule. The microbial stabilizer sorbic acid was integrated as the cores in the product. Potassium sorbate was used for both microbial stabilization and for pH control. The potassium sorbate was introduced into the enzyme layer.

Sorbic acid cores, PSD 150-800 μm, were prepared by sieving in a Russel-Finex C400 800 g cores were loaded into a Glatt Procell AGT100 fluid bed.

A feed for the salt separation layer was produced:

112 g Na2SO4

274 g water at 40-45° C.

The feed was sprayed onto the product in the fluid bed applying the following configuration and process parameters:

Mode of operation: Bottom spray

Nozzle: 1,2 mm

Nozzle air pressure: 1,0 bar

Air flow: 40 m³/hour

Air temperature: 80° C.

Feed flow: 5-15 g/min

Product temp. coating: 40° C.

The enzyme layer was applied in two steps as in example 3.

The feed for the first enzyme layer was produced:

3630 g enzyme concentrate, purified by UF and concentrated to 39,5% DS 114 g Avedex W80

101 g potassium sorbate.

This feed was sprayed onto the product in the fluid bed applying the following configuration and process parameters:

Mode of operation: Bottom spray

Nozzle: 1,2 mm

Nozzle air pressure: 1,5-2,0 bar

Air flow: 40 m³/hour

Air temperature: 80° C.

Feed flow: 12-15 g/min

Product temp. coating: 40° C.

Product temp. drying: 70° C.

2054 g of this product was reloaded into the Glatt Procell GF3 fluid bed.

The feed for the second enzyme layer was produced:

9551 g enzyme concentrate, purified by UF and concentrated to 39,5% DS

314 g Avedex W80

296 g potassium sorbate.

This feed was sprayed onto the product in the fluid bed applying the following configuration and process parameters:

Mode of operation: Bottom spray

Nozzle: 1,2 mm

Nozzle air pressure: 1,2-1,4 bar

Air flow: 70-120 m³/hour

Air temperature: 90° C.

Feed flow: 10-40 g/min

Product temp. coating: 50-54° C.

Product temp. drying: 70° C.

Feed for a final salt layer was produced:

376 g Na₂SO₄

920 g water at 40-45° C.

The feed was sprayed onto the second layer material in the fluid bed applying process conditions as for the salt layer in example 2.

### Example 10

### Incorporation of the pH Controlling Element of the Microbial Stabilizer System in the Top Coat

A salt coated product was made according to the description in example 4. A top coat was applied to this product that included the pH control agent. In this example ascorbic acid was used for the pH control. Ascorbic acid had a surprisingly good effect for control of the dust release properties of the product.

1000 g salt coated product according to example 4 were loaded into a Glatt Procell AGT100 fluid bed.

The feed for the top coat was produced:

271 g Na₂SO₄

271 g ascorbic acid

700 g water at 40-45° C.

This feed was sprayed onto the product in the fluid bed applying the following configuration and process parameters:

Mode of operation: Bottom spray

Nozzle: 1,2 mm

Nozzle air pressure: 1,2-1,4 bar

Air flow: 70 m³/hour

Air temperature: 120° C.

Feed flow: 40 g/min

Product temp. coating: 43-49° C.

### Example 11

### Dextrin Layer Between Core and Enzyme Layer and Sucrose as Binder for the Enzyme Layer

Na₂SO₄ cores from Santa Marta, Na-G1 (coarse) were used

2500 g cores were loaded into a Glatt Procell GF3 fluid bed

A feed for the dextrin separation layer was produced:

63 g dextrin Avedex W 80

188 g water at 40-45° C.

The feed was sprayed onto the product in the fluid bed applying the following configuration and process parameters:

Mode of operation: Bottom spray

Nozzle: 1,2 mm

Nozzle air pressure: 1,8-2,0 bar

Air flow: 90 m³/hour

Air temperature: 90° C.

Feed flow: 7 g/min

Product temp. coating: 59-68° C.

A feed for the enzyme layer was prepared:

9603 g Phytase concentrate (Ronozyme® HiPhos), purified by UF and concentrated to 34 DS

676 g sucrose.

The feed was sprayed onto the cores in the fluid bed applying the following configuration and process parameters:

Mode of operation: Bottom spray

Nozzle: 1,2 mm

Nozzle air pressure: 2,0 bar

Air flow: 110-140 m³/hour

Air temperature: 80° C.

Feed flow: 15-30 g/min

Product temp. coating: 58-63° C.

A feed for the salt layer was produced:

423 g Na₂SO₄

1041 g water at 40-45° C.

The feed was sprayed onto the product in the fluid bed applying the following configuration and process parameters:

Mode of operation: Bottom spray

Nozzle: 1,2 mm

Nozzle air pressure: 2,0 bar

Air flow: 140 m³/hour

Air temperature: 90° C.

Feed flow: 40-95 g/min

Product temp. coating: 50-63° C.

Product temp. drying: 60° C.

### Example 12

### Salt Layer Coated Granule Comprising xylanase

The enzyme in this example was a xylanase (Ronozyme® WX), and the basic product design was similar to the product described in example 2. The example describes a dosage control of the binder for optimal control of the balance between the binding of the enzyme and the formation of large aggregates of agglomerated particles. A good binding is required for a good process yield and a low dust release from the product.

Na₂SO₄ cores, PSD 250-450 μm, were prepared by sieving in a Russel-Finex C400 2500 g cores were loaded into a Glatt Procell GF3 fluid bed.

The enzyme concentrate was purified by UF and concentrated to 20,2% DS. The binder was dextrin Avedex W 80. Four feed solutions were prepared:

| | Feed 1 | Feed 2 | Feed 3 | Feed 4 |
|---|---|---|---|---|
| Enzyme concentrate | 308 g | 928 g | 6220 g | 11888 |
| Binder | 186 g | 189 g | 620 g | 600 g |

The feeds were sprayed onto the cores in the fluid bed applying the following configuration and process parameters:

Mode of operation: Bottom spray

Nozzle: 1,2 mm

Nozzle air pressure: 1,2-1,4 bar

Air flow: 90 gradually increased to 140 m³/hour

Air temperature: 85° C. gradually increased to 100° C.

Feed flow: 10 gradually increased to 42 g/min

Product temp. coating: 59-62° C.

A feed for the salt layer was produced:

364 g Na₂SO₄

891 g water at 40-45° C.

The feed was sprayed onto the product in the fluid bed applying the following configuration and process parameters:

Mode of operation: Bottom spray

Nozzle: 1,2 mm

Nozzle air pressure: 1,2 bar

Air flow: 150 m³/hour

Air temperature: 100° C.

Feed flow: 30-55 g/min

Product temp. coating: 50-63° C.

Product temp. drying: 70° C.

### Example 13

### Salt Layer Coated Granule Comprising Protease

The product design was similar to example 2, and similar process conditions were applied.

Na₂SO₄ cores, PSD 250-350 μm, were prepared by sieving in a Russel-Finex C400 2500 g cores were loaded into a Glatt Procell GF3 fluid bed.

The feed for the enzyme layer was prepared:

11976 g Protease concentrate (Ronozyme® Proact), purified by UF and concentrated to 40,7 DS

682 g dextrin Avedex W 80.

The feed for the salt layer was produced:

366 g Na₂SO₄

896 g water at 40-45° C.

### Example 14

### Low Sucrose Dosage

Similar product recipe and process conditions as in Example 11, but the enzyme containing layer had lower sucrose dosage compared to Example 11:

10100 g Phytase concentrate (Ronozyme® HiPhos), purified by UF and concentrated to 34 DS

505 g sucrose.

### Example 15

### Dextrin as Binder

Similar product recipe and process conditions as in Example 11, but the enzyme containing layer had dextrin Avebe W80 dosage instead of sucrose as binder:

12075 g Phytase concentrate (Ronozyme® HiPhos), purified by UF and concentrated to 34 DS

1330 g dextrin Avebe W80.

1-15. (canceled)

16. A method of applying an enzyme to feed comprising:
preparing a granule comprising a core and at least one enzyme, wherein the enzyme is distributed in the core and/or layered over the core,
dissolving the granule in water, and
applying the liquid onto the feed.

17. The method of claim 16, wherein the granule comprises a coating on the core or an outer layer coating on the granule.

18. The method of claim 16, wherein the dissolved granule is applied onto pellets or mash feed as a liquid composition

19. The method of claim 16, wherein the dissolved granule is applied onto pellets or mash feed as a liquid composition by spray.

20. The method of claim 16, wherein the granule is a layered granule.

21. The method of claim 16, wherein the granule comprises a core and one or more enzyme-comprising layers, wherein the enzyme-comprising layer comprises an enzyme and a binder e.g. a carbohydrate.

22. The method of claim 16, wherein the granule has an outer coating over the enzyme-comprising layer.

23. The method of claim 16, wherein the granule further comprises a microbial stabilizer.

24. The method of claim 16, wherein the granule further comprises a microbial stabilizer, wherein the microbial stabilizer in the granule is present in the core, the enzyme-containing layer, the outer coating, or any combination thereof.

25. The method of claim 16, wherein the granule further comprises a microbial stabilizer, wherein the microbial stabilizer is selected from the group consisting of sorbic acid, ascorbic acid, citric acid, benzoic acid, a salt of sorbic acid, a salt of ascorbic acid, a salt of citric acid, a salt of benzoic acid, potassium sorbate, sodium citrate, sodium benzoate and combinations thereof.

26. The method of claim 16, wherein the granule further comprises a microbial stabilizer, wherein the microbial stabilizer is selected from the group consisting of benzoic acid, sorbic acid, a salt of benzoic acid, a salt of sorbic acid and combinations thereof.

27. The method of claim 16, wherein the material in the core is an inert material and/or a microbial stabilizer, wherein
the inert material is selected from the group consisting of sodium sulfate, sodium chloride, sodium carbonate, sodium nitrate, sodium phosphate, sodium hydrogen phosphate, ammonium sulfate, ammonium chloride, ammonium carbonate, ammonium nitrate, ammonium phosphate, ammonium hydrogen phosphate, potassium sulfate, potassium chloride, potassium carbonate, potassium nitrate, potassium phosphate, potassium hydrogen phosphate, magnesium sulfate, zinc sulfate, sodium citrate, a sugar, a carbohydrate (such as e.g. sucrose, dextrin, glucose, lactose or sorbitol) and combinations thereof, and
the microbial stabilizer is selected from the group consisting of: sorbic acid, ascorbic acid, citric acid, benzoic acid, a salt of sorbic acid, a salt of ascorbic acid, a salt of citric acid, a salt of benzoic acid, potassium sorbate, sodium citrate, sodium benzoate and combinations thereof.

28. The method of claim 16, wherein the enzyme is selected from the group consisting of amylase, beta-glucanase, glycose oxidase, muramidase, phospholipase, phytase, protease, xylanase, and mixtures thereof.

29. A process for producing an enzyme granule for applying enzyme to feed, comprising
preparing a granule comprising a core and at least one enzyme wherein the enzyme is distributed in the core and/or layered over the core, and
applying to the core or layered granule an outer layer to obtain a coated granule, wherein total dust is below 1000 μg/g when measured in the Heubach Type 1 assay and/or below 1000 μg/g when measured in Elutriation assay or wherein active dust fraction is below 20 ppm when measured in the Heubach Type 1 assay and/or below 80 ppm when measured in Elutriation assay.

30. The process of claim 29, wherein the granule is prepared in a fluid bed apparatus.
